(19) United States
(12) Patent Application Publication
(10) Pub. No.: US 2012/0003738 A1
(43) Pub. Date: Jan. 5, 2012

(54) Method For Amplification And Functional Enhancment Of Blood Derived Progenitor Cells Using A Closed Culture System

(75) Inventors: Marco A. Costa, Pepper Pike, OH (US); Masakazu Ishikawa, Shaker Heights, OH (US); Takayuki Asahara, Tokyo (JP); Haruchika Masuda, Kanagawa (JP)

(73) Assignee: University Hospitals of Cleveland

(21) Appl. No.: 13/164,392

(22) Filed: Jun. 20, 2011

Related U.S. Application Data
(63) Continuation-in-part of application No. 11/884,949, filed on Oct. 4, 2007.

(51) Int. Cl. C12N 5/078; C12N 5/02

(52) U.S. Cl. 435/372; 435/325; 435/406

(57) ABSTRACT

The present invention provides a method for expanding and improving functional capacity of human adult-derived progenitor cells in vitro using a closed culture system. The present invention provides a favorable condition for cell therapy to promote tissue repair and organogenesis via vasculogenesis and angiogenesis in clinical settings. The proposed closed bag culture system for culturing hemangioblast comprises of, in one embodiment, a serum-free culture medium containing one or more factors selected from the group consisting of stem cell growth factor, interleukin-6, FMS-like tyrosine kinase 3, thrombopoietin, and vascular endothelial growth factor and a kit for the preparation of the serum-free culture medium and the like.

### CROSS-REFERENCE TO RELATED APPLICATIONS

This application is a continuation-in-part of U.S. patent application Ser. No. 11/884,949, filed on. Feb. 22, 2006, which is incorporated herein by reference [1].

### FIELD OF THE INVENTION

The present invention relates to a method for culturing hemangioblasts, CD-34+ cells, CD-133+ cells, or unselected mononuclear cells obtained by culturing in a non-serum-containing medium with cytokines using closed bag culture system and the like. These cultured or expanded cells can be used for therapeutic applications not only targeting cardiovascular diseases but also applied to the repair musculoskeletal and neurological diseases.

### BACKGROUND OF THE INVENTION

Bone marrow derived mononuclear cell transplantation therapy and a cell transplantation using CD-34+ cells by collecting peripheral-blood stem cells have been applied in recent years. However, some problems such as those mentioned below have been identified:

1) Any existing therapy causes physical burden and risks on patients, such as general anesthesia, prolonged administration of granulocyte colony stimulating factor (G-CSF), need of central vein access, apheresis, bone-marrow aspiration and the like.

2) Repetitive cell transplantation therapy is difficult using such methods.

3) Treatment of acute illness such as stroke, heart attack, muscle or bone injuries is unsafe and cumbersome using such methods

4) Supply of progenitor cells in adult humans, both qualitatively and quantitatively, is insufficient for therapeutic applications, particularly in patients with chronic diseases.

5) Cells obtained from patients with chronic or acute illness are also defective.

6) Conventional dish or open-system based cell culture approaches are complex and requires special expertise, limits large scale application and there is higher risk of contamination.

7) Special expertise, a tertiary care medical center and costly infrastructure is required for bone-marrow aspiration, apheresis, and conventional cell culture systems.

8) Transportation of cultured/expanded/enhanced cells to different geographic locations for treatment of needed populations, or to treat war related injury at remote locations is not feasible with conventional open system culture approaches.

A system and method are needed which provide for the economic, transportable, safe, effective and consistent amplification of endothelial progenitor cells.

### SUMMARY OF THE INVENTION

An object of the present invention is to provide a method for expanding functional undifferentiated blood derived CD-34+ cells or unselected mononuclear cells (MNC) in vitro for cell transplantation in humans with acute and chronic cardiac, vascular, neurological and musculoskeletal diseases, and provide a safer, and more feasible and cost-effective approach clinical-associated culture system obtained by the method. In the view of the above-mentions problems, the present inventors have studied cultivation conditions permitting undifferentiated endothelial progenitor cells to differentiate and expand in vitro using closed culture system using dedicated reservoir (bag, tube, or container). As a result, the present inventors have succeeded in efficient expansion of CD-34+ cells in vitro by, in one embodiment, culturing a hemangioblast in a serum-free culture medium comprising (1) a stem cell factor (SCF), (2) interleukin-6 (IL-6), (3) FMS-like tyrosine kinase 3 (Flt-3) and (4) thrombopoietin (TPO), and for greater angiogenic potential in vivo of CD-34+ cells by further adding, in one embodiment, (5) a vascular endothelial growth factor (VEGF) to the medium and the like. The present inventors have also succeeded in efficient expansion of MNC in vitro by culturing a hemangioblast in this serum-free culture medium. Moreover, a closed bag culture system provides more therapeutic potential of cells and more feasible procedure invention in practical clinical settings, which resulted in the completion of the present.

The invention relates in one embodiment to a method for expanding and improving the functional capacity of human adult-derived progenitor cells (hemangioblasts) or MNC in vitro using, in one embodiment, a closed bag culture system that promotes vasculogenesis and angiogenesis for tissue repair and organogenesis. The closed bag culture system comprises serum-free culture medium containing one or more factors selected from the group consisting of stem cell growth factor, interleukin-6, FMS-like tyrosine kinase 3 and thrombopoietin. Proposed uses for this system include expanding functional undifferentiated CD-34+ cells, CD-133+ cells, and MNC in vitro for cell transplantation in humans with acute and/or chronic cardiac, vascular, neurological and musculoskeletal diseases, as well as providing safer, more feasible and cost-effective approaches to current clinical-associated culture systems. The key advancement of this closed system is that it prevents complications from infection, cell preparation at clinical sites, obviating the need of highly specialized cell transplant center or laboratory, and enables convenient transport of the cells.

In the view of the above-mentions problems, the present inventors studied cultivation conditions that permit undifferentiated endothelial progenitor cells to differentiate and expand in vitro using a dedicated reservoir (bag, tube, container, etc). These experiments revealed efficient expansion of CD-34+ cells, CD-133+ cells, and MNC in vitro by culturing hemangioblasts in serum-free culture medium containing (1) a stem cell factor (SCF), (2) interleukin-6 (IL-6), (3) FMS-like tyrosine kinase 3 (Flt-3) and (4) thrombopoietin (TPO). Greater CD-34+ cells, CD-133+ cells, and MNC angiogenic potential in in vivo applications was achieved by adding (5) vascular endothelial growth factor (VEGF) to the medium. Previous work from Asahara in Japan [1] did not compare 4 cytokines versus 4 plus VEGF—hence, the current invention includes such a comparison (FIG. 3), including in vivo experiments (FIG. 4).

In one embodiment the invention relates to a method for expanding a hemangioblast, comprising incubating the hemangioblast in serum-free culture medium containing stem cell factor, interleukin-6, FMS-like tyrosine kinase 3 and thrombopoietin in a closed culture system. In one embodiment the invention relates to a method for expanding a hemangioblast, comprising incubating the hemangioblast in serum-free culture medium containing stem cell factor, interleukin-6, FMS-like tyrosine kinase 3, thrombopoietin, and vascular endothelial growth factor in a closed culture system. In one embodiment said closed culture system is selected from the group consisting of a bag, tube, flask, plate, and vessel. In one embodiment said closed culture system contains resealing access ports which provide closed growth environment with sterile fluid path, thereby reducing risk of contamination. In one embodiment said closed culture system is exemplified by such culture vessels as Corning® RoboFlask™, CELLSTAR® AutoFlask™, OptiCell™, and Petaka™ cell culture devices and the like. In one embodiment, the hemangioblast is derived from bone marrow, cord blood or peripheral blood. In one embodiment, the hemangioblast is a mononuclear cell (MNC). In one embodiment, the hemangioblast is CD34 positive and CD133 positive. In one embodiment, the hemangioblast and serum-free culture medium are derived from animals of the same species. In one embodiment the hemangioblast is derived from human. In one embodiment the serum-free culture medium further comprises a vascular endothelial growth factor and a transforming growth factor β inhibitor. In one embodiment, the serum-free culture medium further comprises a transforming growth factor β inhibitor. In one embodiment the serum-free culture medium further comprises a vascular endothelial growth factor. In one embodiment, the invention is an endothelial progenitor cell obtained by the method described above. In one embodiment, the invention is a composition comprising an endothelial progenitor cell obtained by the method described above, wherein said cell is substantially free of a biogenic substance derived from an animal of a different species from the animal, from which the endothelial progenitor cell is derived. In one embodiment the endothelial progenitor cell obtained by the method described above is CD34 positive and CD133 positive. In one embodiment, said hemangioblast is obtained from a subject provided: a) administration of granulocyte colony stimulating factor over 3 days, b) conventional extraction of peripheral blood sample from said subject, and c) isolation of desired mononuclear cells by density gradient centrifugation. In one embodiment, said blood sample is 400 milliliters or less in volume. In one embodiment, said subject is a human.

In one embodiment, the invention is a kit for preparing a serum-free culture medium containing a stem cell factor (SCF), interleukin-6 (IL-6), FMS-like tyrosine kinase 3 (Flt-3) and thrombopoietin (TPO) in a closed culture system. In one embodiment, the invention is a kit for preparing a serum-free culture medium containing a stem cell factor (SCF), interleukin-6 (IL-6), FMS-like tyrosine kinase 3 (Flt-3) and thrombopoietin (TPO), and for a greater angiogenic potential in in vivo applications of CD-34+ cells further adding a vascular endothelial growth factor (VEGF) to the serum-free medium in a closed culture system. In one embodiment, the invention is a kit for preparing a serum-free culture medium containing a stem cell factor (SCF), interleukin-6 (IL-6), FMS-like tyrosine kinase 3 (Flt-3), thrombopoietin (TPO), and vascular endothelial growth factor (VEGF) in a closed culture system.

In one embodiment the invention comprises a method for expanding a hemangioblast population, comprising incubating hemangioblasts in serum-free culture medium, said medium comprising stem cell factor, interleukin-6, FMS-like tyrosine kinase 3, thrombopoietin, and vascular endothelial growth factor in a closed culture system under conditions such that the number of hemangioblasts increases. In one embodiment closed culture system is selected from the group consisting of a bag, tube, flask, plate, and vessel. In one embodiment said closed culture system contains resealing access ports which provide closed growth environment with sterile fluid path, thereby reducing risk of contamination. In one embodiment said hemangioblasts are derived from bone marrow, cord blood or peripheral blood. In one embodiment said hemangioblast is a mononuclear cell. In one embodiment said hemangioblast is CD34 positive and CD133 positive. In one embodiment the hemangioblast and serum-free culture medium are derived from animals of the same species. In one embodiment the hemangioblasts are human hemangioblasts. In one embodiment said serum-free culture medium further comprises a transforming growth factor β inhibitor. In one embodiment the invention comprises an endothelial progenitor cell obtained by the method of described above.

In one embodiment said hemangioblast is obtained from a subject treated with: a) granulocyte colony stimulating factor over 3 days or less. In one embodiment, following said treating, a peripheral blood sample is obtained from said subject. In one embodiment said said blood sample is subjected to density gradient centrifugation in order to obtain said hemangioblasts. In one embodiment said blood sample is 400 milliliters or less in volume. In one embodiment said subject is a human.

In one embodiment the invention comprises a composition comprising an endothelial progenitor cell obtained by the method described above, wherein said cell is substantially free of a biogenic substance derived from an animal of a different species from the animal, from which the endothelial progenitor cell is derived.

In one embodiment the invention comprises a kit for preparing a serum-free culture medium, said kit comprising stem cell factor, interleukin-6, FMS-like tyrosine kinase 3, thrombopoietin, vascular endothelial growth factor, and serum-free culture medium in a closed culture system.

In one embodiment the invention comprises a method for culturing a hemangioblast, comprising incubating the hemangioblast in a closed culture system in serum-free culture medium containing stem cell factor, interleukin-6, FMS-like tyrosine kinase 3, thrombopoietin, and vascular endothelial growth factor.

In one embodiment the invention comprises a method of treating a blood donor in order to obtain hemangioblasts from said donor provided: a) treating said donor with granulocyte colony stimulating factor over the course of 3 or less days, b) extracting a peripheral blood sample from said donor after the course of treatment, and c) isolating desired mononuclear cells by density gradient centrifugation. In one embodiment said blood sample is 400 mL or less in volume. In one embodiment said subject is a human.

In one embodiment, the invention is a method for culturing a hemangioblast, comprising incubating the hemangioblast in a closed culture system in serum-free culture medium containing stem cell factor, interleukin-6, FMS-like tyrosine kinase 3 and thrombopoietin. An open cell culture system is essentially limited to basic science laboratories or pre-clinical animal experiments, and the use of its cell product to treat diseases in humans faces significant logistical and regulatory challenges making this approach unlikely to be of any clinical value. The advantages of a closed culture system compared with a bench top, open cell culture system have been described above. While providing similar culture conditions and expansion capability, a closed system allow cell processing to be performed without the need for highly specialized cell culture Hoods, cells do not enter in contact with open air, minimizes risk of infection, allows transportation from a centralized lab to different geographic regions for treatment, may enable cell preparation or transport to remote sites of war or troops deployment to treat injuries at the site. In another embodiment, the invention is a method for culturing a hemangioblast, comprising incubating the hemangioblast in a closed culture system in serum-free culture medium containing stem cell factor, interleukin-6, FMS-like tyrosine kinase 3, thrombopoietin, and vascular endothelial growth factor.

In another embodiment, the cultivation method of the present invention enables expansion of hemangioblast populations provided: a) granulocyte colony stimulating factor over 3 days or less (i.e. preferably not more than 3 days), b) conventional extraction of peripheral blood sample from a patient, c) isolated of desired mononuclear cells by density gradient centrifugation d) mononuclear cell culture with serum-free expansion medium in a closed system previously described. In one embodiment the blood sample is 400 milliliters or less in volume.

In one embodiment, the product is derived after CD34+ expansion and comprises a population composed of cells wherein >50% express the CD34+, and have upregulation of HGF and mir-210 (micro-RNA 210). In another embodiment, the cells are devoid of macrophage/monocyte or lymphocyte markers (not inflammatory or immunologic cells) based on fluorescence-activated cell sorting (FACS) data seen in (FIG. 6B).

In one embodiment, the invention relates to the expansion of unselected blood derived mononuclear cells (MNC). This is key and novel step and saves a major step in the cell processing product as it obviates the need to filter or select CD34+ cells. Isolation/selection/filtering of CD34+ cells is somewhat problematic because there are currently no low volume cell sorting systems that can be used clinically in the United States.

The cell product of the unselected blood derived MNC 7 day expansion using the same media is composed of CD34+ cells (usually <20%), CD3+/CD31+ cells (20-4-%), but the system also expands CD3+/CD31+/CXCR4+ cells—these are known as pro angiogenic T cells. The expansion media of the current invention, similarly to the CD34+ cells isolation process, promotes up-regulation of HGF, angiopoietin-2 and mir-210—all associated with pro-angiogenesis.

The expansion process of one embodiment of the method of the current invention for MNC is shorter, 3 days (or less) instead of 7 days. This expansion time period favors CD3/CD31/CXCR4 positive cell expansion with a 10-15 fold increase in number of these specific cells.

The present invention is explained in more detail in the following by referring to the Examples, which are described for explanation of the present invention and do not limit the present invention in any way.

### BRIEF DESCRIPTION OF THE DRAWINGS

For a more complete understanding of the features and advantages of the present invention, reference is now made to the detailed description of the invention along with the accompanying figures.

FIG. 1 shows a schematic comparison of the current approach used in the ACT-34 trial for CD34⁺ cell therapy and the approach of the current invention (also referred to as the StemMed West approach).

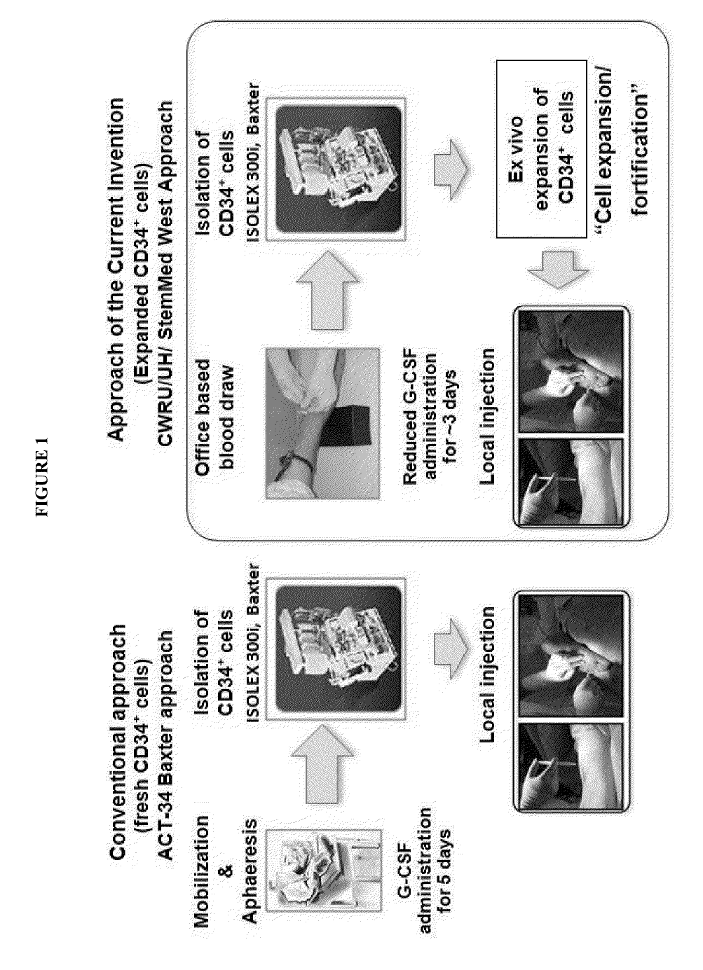

FIG. 2 shows (A): the concept of ex vivo expansion culture of the current invention. (B): Scheme of current invention (StemMed West) serum-free expansion culture medium (current invention). (C): Picture of the current invention (StemMed West) Expansion platform (current invention). Sterile closed culture bag or cassette system for simple and feasible culture in clinical settings (current invention).

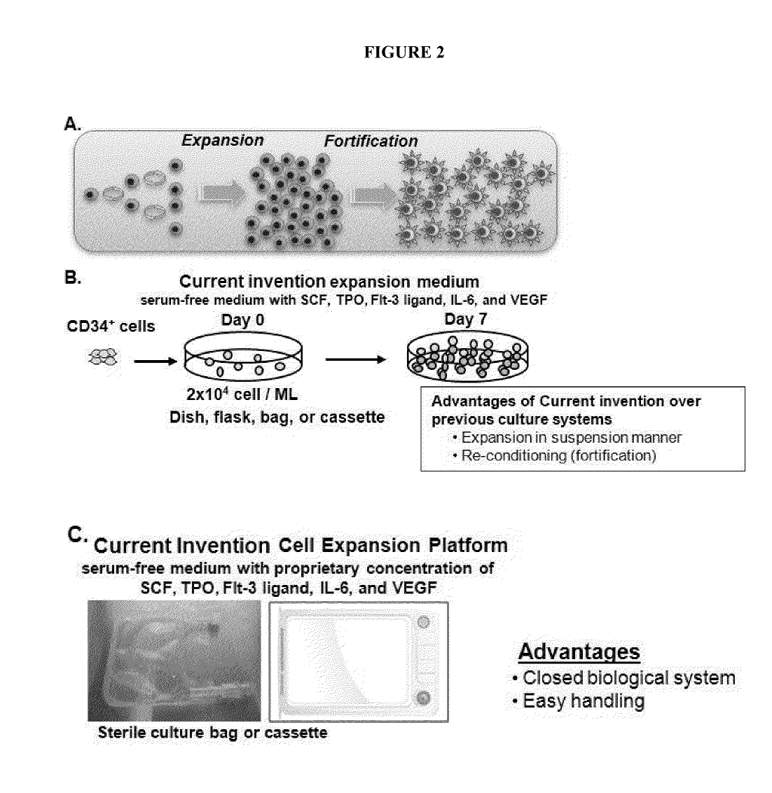

FIG. 3 shows (A): Increase in cell number 7 days after culture of umbilical cord blood CD34⁺ cells. Graph showed about 18 fold amplification of cell number in our complete medium (postEX). There is no significant difference between our complete culture medium and 4 factors without VEGF (postEX without VEGF). (B): CD34 positivity after expansion culture was decreased in both of the postEX and postEX without VEGF groups around 45%. (C): miR-210, pro-angiogenic microRNA, was significantly upregulated in the postEX group. (D): Representative tube formation assay images for evaluation of angiogenic potential of cells in vitro in each group. Bar graph showed significant increase in number of branch points in the postEX group compare to other groups. In the postEX without VEGF group, there was no significant increase. Interestingly, CD34⁺ cells expanded with the method of the current invention lost angiogenic potential after miR-210 silencing.

FIG. 4 shows (A) Representative laser Doppler Images for each group on days 0 and 14 after treatment showing increased flow in the left hind limbs (target) of the postEX group. (B): Graphic showing significantly increased mean flux ratio in the postEX group. There was no significant increase in the postEX with miR-210 silencing and postEX without VEGF groups. (C): Representative images of immunofluorescent staining with CD31 antibody using transverse sections of calf muscles in each group at day 14. Capillaries were shown in green (original magnification, 200×; scale bar=100 μm). (D): Capillary density was significantly enhanced in the postEX group compared to other groups (*P<0.05, **P<0.01).

FIG. 5 shows representative samples of preliminary tissue histopathological evaluation of showing the lack of tumor formation after treatment with phosphate buffered saline (PBS) versus CD34⁺ cells pre- and post-EX using the media of the current invention (StemMed West).

FIG. 6 shows (A): Increase in cell number 7 days after culture of mobilized peripheral blood CD34⁺ cells. Graph showed about 8 fold amplification in cell number. (B): Flow cytometry data showed >40% CD34 positivity in postEX CD34⁺ cells. (C): Representative tube formation assay images in each group. (D): Bar graph showed significant increase in number of branch points in the postEX group compared to PBS and preEX groups (n=6 in each group, **P<0.01).

FIG. 7 shows (A) Semi-quantitative measurement of gross tissue damage and gait function after treatment with same doses of each group at day 14 using scoring sheet [2]. Graphic showing significant tissue preservation in animals treated with postEX CD34⁺ cells. There was trend toward lower score of gait function in postEX group (n=12 in each group, *P<0.05). (B) Representative laser Doppler Images for each group on days 0 and 14 after treatment showing increased flow in the left hind limbs (target) of animals injected with postEX cells. Amputation rate after treatment with postEX cells was reduced (n=12 in each group). (C): Graphic showing significantly increased mean flux ratio in the postEX group, although there was no significant difference between the PBS and preEX groups (n=12 in each group, *P<0.05, *P<0.01).

FIG. 8 shows (A): Representative images of immunofluorescent staining with CD31 antibody using transverse sections of adductor muscles in each group at day 7. Capillaries were shown in green (original magnification, 200×; scale bar=100 μm). (B): Capillary density was significantly enhanced in the postEX group compared to the PBS and preEX groups (*P<0.05, **P<0.01).

FIG. 9 shows a representative double-immunofluorescence staining for human mitochondria (hMit) and CD31 at day 14 in each group using transverse section of adductor muscle. Human endothelial cells were identified as double-positive cells for hMit (red) and CD31 (green) (arrows). The number of double-positive cells was scarce in the preEX group. There were no double-positive cells in the PBS group (original magnification, 400×; scale bar=100 μm).

FIG. 10 shows real-time PCR analysis for intrinsic angiogenic markers at days 3 and 7. All angiogenic factors were significantly up-regulated in the postEX group at day 3 in mouse adductor muscle. There was a similar trend in calf muscle except ANG2. Gene expressions were enhanced only in the early phase after surgery (n=3 in each group, *P<0.05, **P<0.01).

FIG. 11 shows mononuclear cell culture with our serum-free expansion medium. Healthy adult non-mobilized or mobilized mononuclear cells (nmMNC and mMNC, respectively) were cultured with our expansion medium. At days 3 and 7 after culture, cells were evaluated.

FIG. 12 shows cell number and flow cytometric data after 3-day culture of non-mobilized mononuclear cells. (A and B): Number of total MNCs was decreased around 50% of preEX at 3 days (n=4, **p<0.01). (C): Flow cytometry data showing no significant increase or decrease of CD34 positivity at day 3. (D and E): Flow cytometry data showing significant increase of CD3⁺/CD31⁺ and CD3⁺/CD31⁺/CXCR4⁺ cells, “angiogenic T cells” (n=4, *p<0.05, **p<0.01).

FIG. 13 shows absolute cell numbers from flow cytometry data after 3-day culture of non-mobilized mononuclear cells. (A-F): The number of CD34⁺ cells and CD3⁺/CD31⁺ cells was significantly decreased after 3-day culture, however, CD3⁺/CD31⁺/CXCR4⁺ cells were significantly amplified (n=4, *p<0.05, **p<0.01).

FIG. 14 shows (A): Real-time PCR analysis for angiogenic makers of 3-day cultured MNCs with the medium of the current invention. Interestingly, only ANG-2 expression was significantly upregulated in the postEX group (n=4 in each group, **P<0.01). (B): miR-210, pro-angiogenic microRNA, was significantly upregulated in the postEX group (n=5 in each group, **P<0.01).

FIG. 15 shows the cell number after culture of mobilized mononuclear cells with our medium for 7 days. (A-D): Total mMNC numbers were decreased around 0.3 fold after 7 days culture. (E-H): On the other hand, CD34⁺ cells were amplified around 3 fold after 7 days culture of mMNCs.

FIG. 16 shows an embodiment of the current invention, a method for amplification of stem/progenitor cells using easy handling culture devices. Sterile closed culture bag or cassette system for simple and feasible culture would be favorable in clinical settings.

FIG. 17 shows (A): Umbilical cord blood CD34⁺ cells were cultured with our medium using conventional plate (6-well plate) or closed bag for 7 days. Total cell number after culture showing trend toward to lower number in bag culture. (B): Representative flow cytometry data from both of plate and bag culture. (C): There was no significant difference in CD34 positivity between plate and bag culture.

FIG. 18 shows (A): Gene expression of HGF (hepatocyte growth factor) was significantly upregulated in bag cultured CD34⁺ cells (n=3 in each group, **P<0.01). (B): In addition, miR-210, pro-angiogenic microRNA, was significantly upregulated in bag cultured CD34⁺ cells (n=3 in each group, **P<0.01).

FIG. 19 shows a scheme of current invention approach.

FIG. 20 shows the confirmed CD34⁺ cell therapeutic potential in various kinds of pre-clinical animal models. Therefore, the present invention will yield various products in cardiovascular, orthopedics [3, 4], and wound-care [5-8] as natural extensions of the technology.

Table 1 shows features and clinical limitations of current CD34⁴ cell therapy approach compared with the approach of the current invention (CWRU/UH/StemMed West).

### DEFINITIONS

To facilitate the understanding of this invention a number of terms are defined below. Terms defined herein (unless otherwise specified) have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, terms defined in the singular are intended to include those terms defined in the plural and vice versa.

Endothelial progenitor cells are a population of rare cells that circulate in the blood with the ability to differentiate into endothelial cells, the cells that make up the lining of blood vessels. The process by which blood vessels are born de novo from endothelial progenitor cells is known as vasculogenesis. Most of vasculogenesis occurs in utero during embryologic development. Endothelial progenitor cells, were therefore first believed to be angioblasts, which are the stem cells that form blood vessels during embryogenesis. Endothelial progenitor cells participate in pathologic angiogenesis such as that found in retinopathy and tumor growth. While embryonic angioblasts have been known to exist for many years, adult endothelial progenitor cells were first believed to be characterized in the 1990s after Asahara and colleagues published that a purified population of CD34-expressing cells isolated from the blood of adult mice could purportedly differentiate into endothelial cells in vitro [9].

It is also known that various cytokines, growth factors, and hormones cause hematopoietic cells, and by association endothelial progenitor cells, to be mobilized into the peripheral circulation, ultimately homing to regions of angiogenesis [10].

A hemangioblast is a multipotent cell, common precursor to hematopoietic and endothelial cells [11]. Hemangioblasts have been first extracted from embryonic cultures and manipulated by cytokines to differentiate along either hematopoietic or endothelial route. It has been shown that these pre-endothelial/pre-hematopoietic cells in the embryo arise out of a phenotype CD34 population. It was then found that hemangioblasts are also present in the tissue of fully developed individuals, such as in newborn infants and adults. There is evidence of hemangioblasts that continue to exist in the adult as circulating stem cells in the peripheral blood that can give rise to both endothelial cells and hematopoietic cells. These cells are thought to express both CD34 and CD133 [12]. These cells are likely derived from the bone marrow, and may even be derived from hematopoietic stem cells.

A peripheral blood mononuclear cell (PBMC) is any blood cell having a round nucleus. For example: a lymphocyte, a monocyte or a macrophage. These blood cells are a critical component in the immune system to fight infection and adapt to intruders. The lymphocyte population consists of T cells (CD4 and CD8 positive ˜75%), B cells and NK cells (˜25% combined). These cells are often extracted from whole blood using ficoll, a hydrophilic polysaccharide that separates layers of blood, with monocytes and lymphocytes forming a buffy coat under a layer of plasma. This huffy coat contains the PBMCs. Additionally; PBMC can be extracted from whole blood using a hypotonic lysis which will preferentially lyse red blood cells. This method results in neutrophils and other polymorphonuclear (PMN) cells which are important in innate immune defense being obtained. PBMCs are widely used in research and clinical uses every day. HIV research uses them because PBMCs include CD4+ cells, which are the cells HIV infects.

Hepatocyte growth factor/scatter factor (HGF/SF) is a paracrine cellular growth, motility and morphogenic factor. It is secreted by mesenchymal cells and targets and acts primarily upon epithelial cells and endothelial cells, but also acts on haemopoietic progenitor cells.angiopoietin-2.

Mir-210 is a short RNA molecule.

Granulocyte colony-stimulating factor (G-CSF or GCSF) is a colony-stimulating factor hormone. G-CSF is also known as colony-stimulating factor 3 (CSF 3).

### Experimental

The following are examples that further illustrate embodiments contemplated by the present invention. It is not intended that these examples provide any limitations on the present invention.

In the experimental disclosure that follows, the following abbreviations apply: eq. or eqs. (equivalents); M (Molar); μM (micromolar); N (Normal); mol (moles); mmol (millimoles); μmol (micromoles); nmol (nanomoles); pmoles (picomoles); g (grams); mg (milligrams); μg (micrograms); ng (nanogram); vol (volume); w/v (weight to volume); v/v (volume to volume); L (liters); ml (milliliters); μl (microliters); cm (centimeters); mm (millimeters); μm (micrometers); nm (nanometers); C (degrees Centigrade); rpm (revolutions per minute); DNA (deoxyribonucleic acid); kdal (kilodaltons).

### EXAMPLES

The invention is further described in detail by reference to the following experimental examples. These examples are provided for purposes of illustration only, and are not intended to be limited unless otherwise specified. Thus, the invention should in no way be construed as being limited to the following examples, but rather should be construed to encompass any and all variation which become evident as a result of the teaching provided herein. The materials and methods employed in the experiments are now described.

### Example 1

The study hypothesis was that CD34⁺ cells could be expanded with fortification of angiogenic potential using the current invention (StemMed West) approach and final cell product could promote therapeutic angiogenesis in mouse hind limb ischemia (HLI) compared with fresh CD34⁺ cells (control) and phosphate buffered saline (PBS). To confirm this hypothesis, first we used umbilical cord blood CD34⁺ cells.

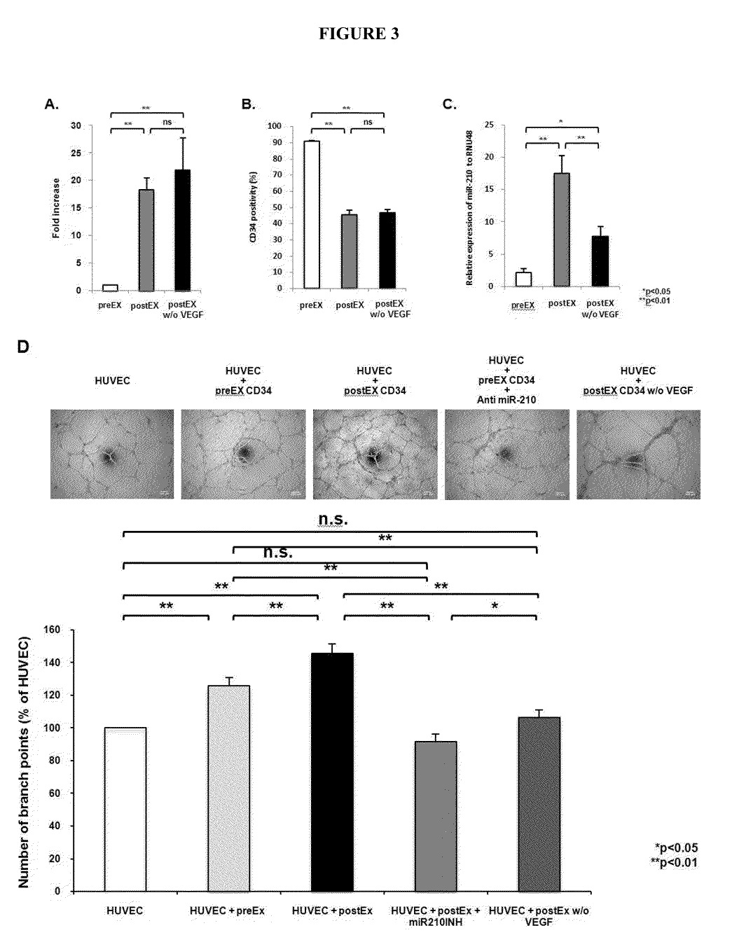

CD34⁺ cells were expanded using the methods and kits of the current invention (StemMed West system) maintaining their CD34-positivity around 45% (FIGS. 3A and B). Although, there was no significant difference between our expansion method (5 cytokines) and 4-cytokine method without VEGF in cell amplification and maintenance of CD34-positivity, expression of miR-210, pro-angiogenic microRNA, was significantly up regulated in expanded CD34⁺ cells with our method (FIG. 3C). In addition, in vitro tube formation assay showed significant increase of the number of branch points in the postEX group compared with the HUVEC and preEX groups. Moreover, cultures with miR-210 silencing and 4-cytokine without VEGF led lower angiogenic potential of cells (FIG. 3D).

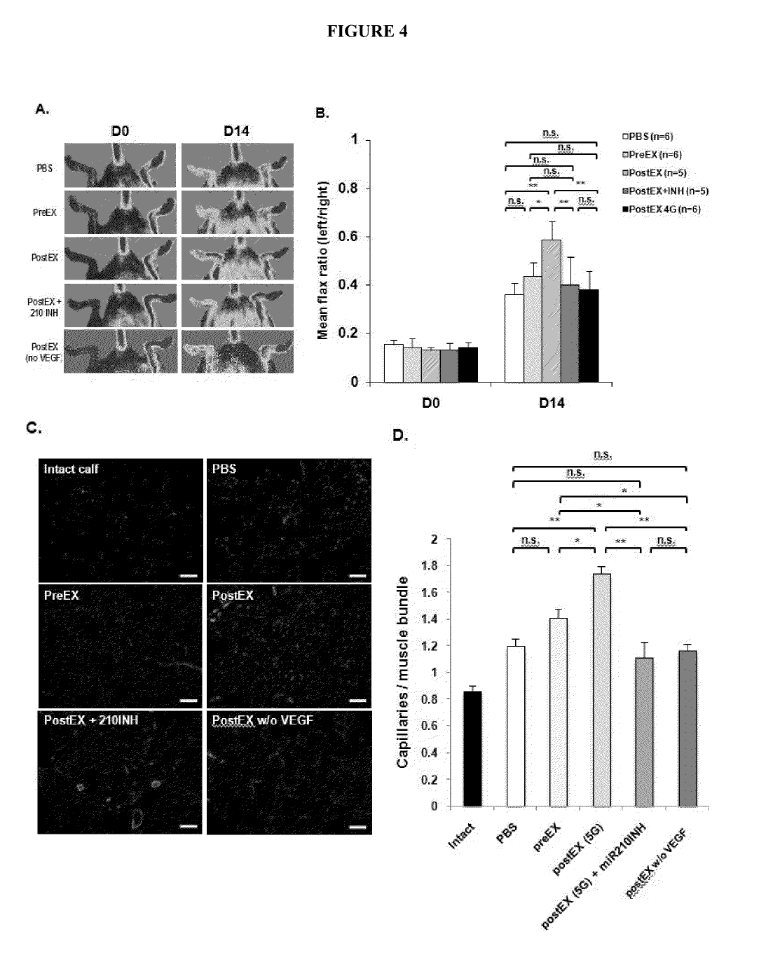

For in vivo study to confirm therapeutic potential of cells, HLI was induced by ligation of femoral artery in 8-12 week old immunodeficient mice (NOD/SCID mouse). Based on the cell dose utilized in the current clinical trial (1×10⁵ cells/kg/limb, ACT34-CLI, Baxter), 2.5×10⁴ CD34⁺ cells/mouse were injected intramuscularly into the affected limb 24 hours after HLI induction. Cell dose and time of therapy were selected to closely replicate the clinical setting and time frame of clinical presentation of patients with CLI. Expanded CD34⁺ cells showed superior therapeutic potential compared to fresh CD34⁺ cells in mouse HLI model: 1) promoted significantly higher blood flow in the affected limb and 2) promoted significantly enhanced angiogenesis in the calf muscle of affected limb compared with other groups (FIG. 4). These results were confirmed in replicate, including a set of experiments with cell treatment performed at the same time of surgery.

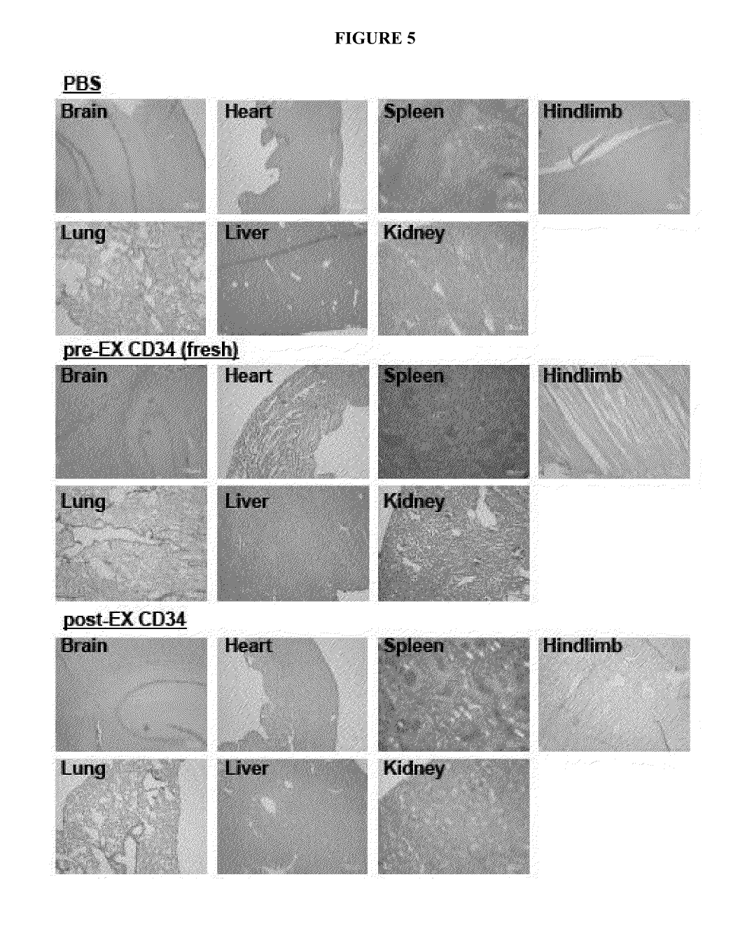

Finally, long-term safety studies were conducted to detect potential tumorigenesis. Histological analysis did not reveal signs of pathological angiogenesis or tumor formation in animals treated with expanded CD34⁺ cells compared with negative control or fresh CD34⁺ cells. FIG. 5 shows representative samples of preliminary tissue histopathological evaluation of showing the lack of tumor formation after treatment with phosphate buffered saline (PBS) versus CD 34⁺ cells pre- and post-EX using the current invention media (StemMed West media).

### Example 2

Following the umbilical cord blood CD34+ cell experiment, we used granulocyte colony-stimulating factor (G-CSF) mobilized adult peripheral blood CD34⁺ cells (GMCD34⁺ cells) to confirm our hypothesis described in EXAMPLE 1. This mPB-CD34⁺ cells are used in the current clinical trial of CD34⁺ cell therapy for critical limb ischemia. Therefore, we chose this fraction, although the umbilical cord blood CD34⁺ cells are potential cell candidate because of their higher therapeutic potential.

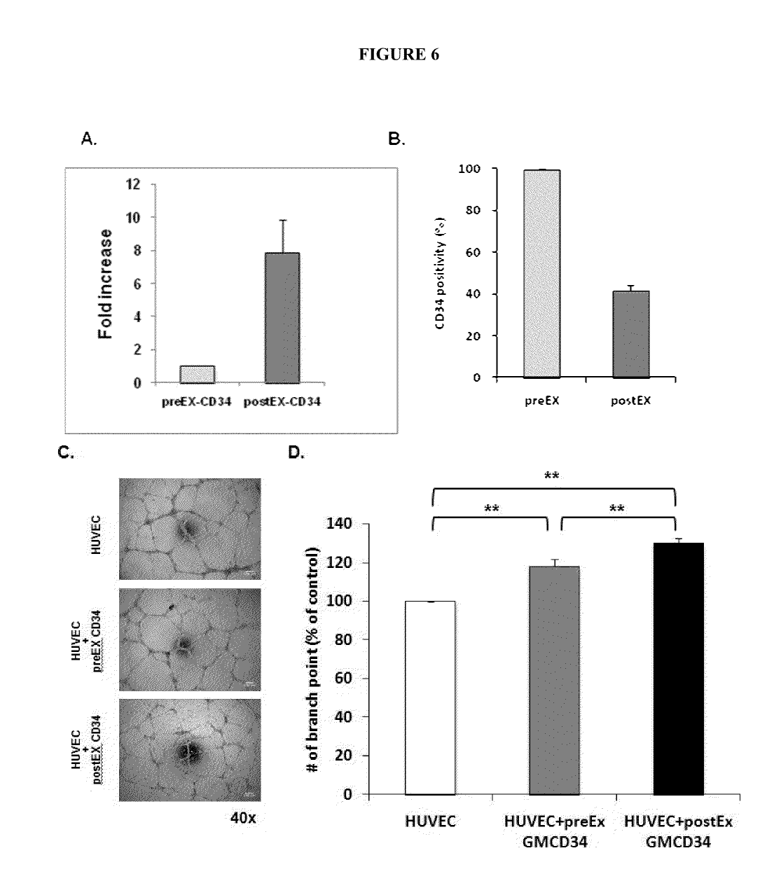

The GMCD34⁺ cells were expanded using the methods and kits of the current invention (StemMed West system) maintaining their CD34-positivity around 40% (FIGS. 6A and B). In vitro tube formation assay showed significant increase of the number of branch points in the postEX GMCD34 group compared with the HUVEC and preEX GMCD34 groups (FIGS. 6C and D).

For in vivo study to confirm therapeutic potential of cells as well as umbilical cord blood CD34⁺ cells, HLI was induced by ligation of femoral artery in 8-12 week old immunodeficient mice (Nude mouse, NCR nu/nu). After 24 hours HLI induction, 2.5×10⁴ CD34⁺ cells/mouse were injected intramuscularly into the affected limb. Cell dose and time were followed as described in EXAMPLE 1 to simulate practical clinical situation.

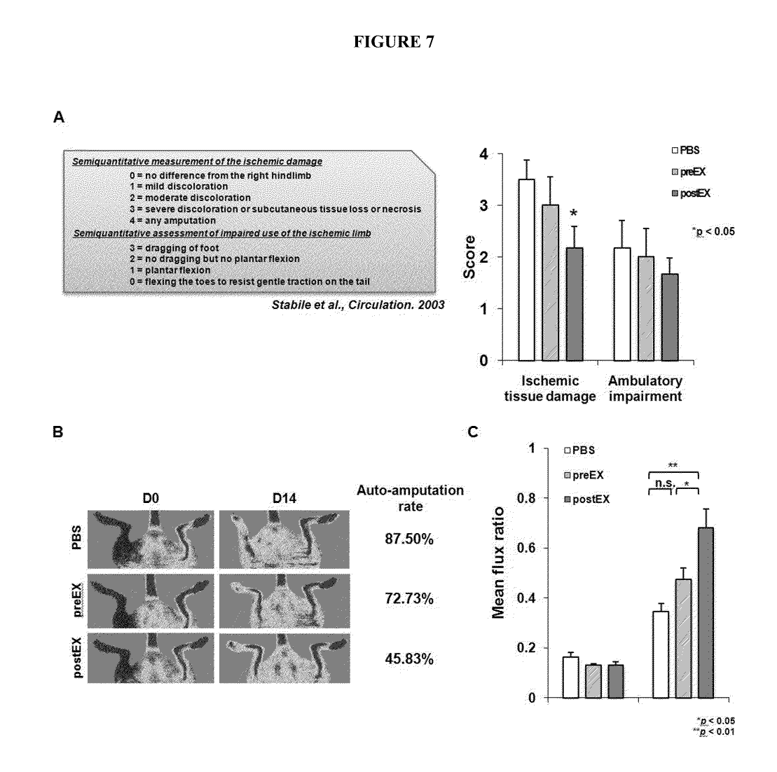

Expanded GMCD34⁺ cells showed superior therapeutic potential compared to preEX-GMCD34⁺ cells in mouse HLI model: 1) significantly improved ischemic tissue damage, 2) reduced amputation rate, and 3) promoted significantly higher blood flow in the affected limb (FIG. 7).

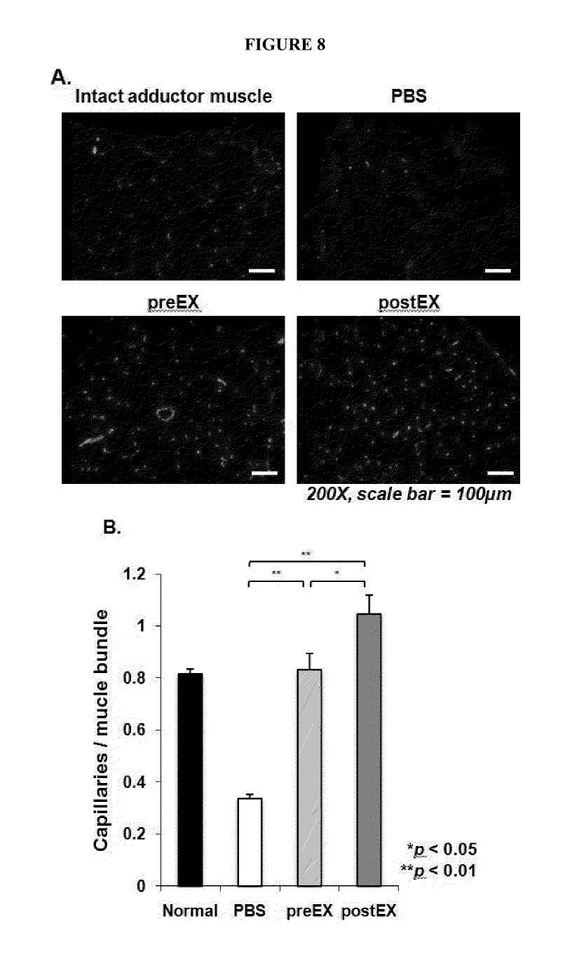

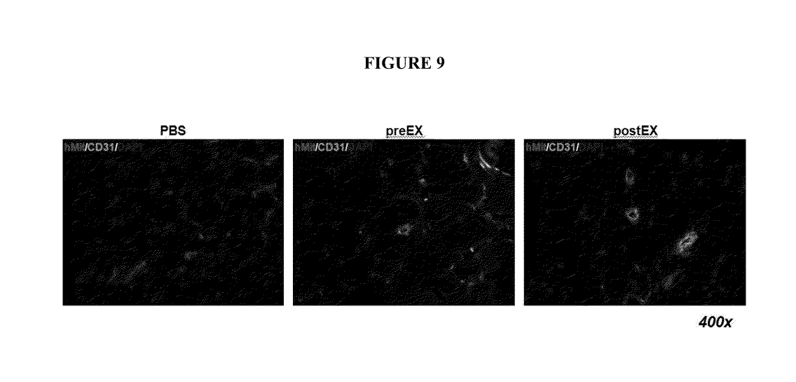

Immunofluorescent staining revealed significantly enhanced capillary density in the adductor muscle after postEX-GMCD34⁺ cells compared to other groups (FIG. 8). And it was demonstrated that injected human preEX and postEX-GMCD34⁺ cells were differentiated into endothelial cells in mouse adductor muscle (FIG. 9).

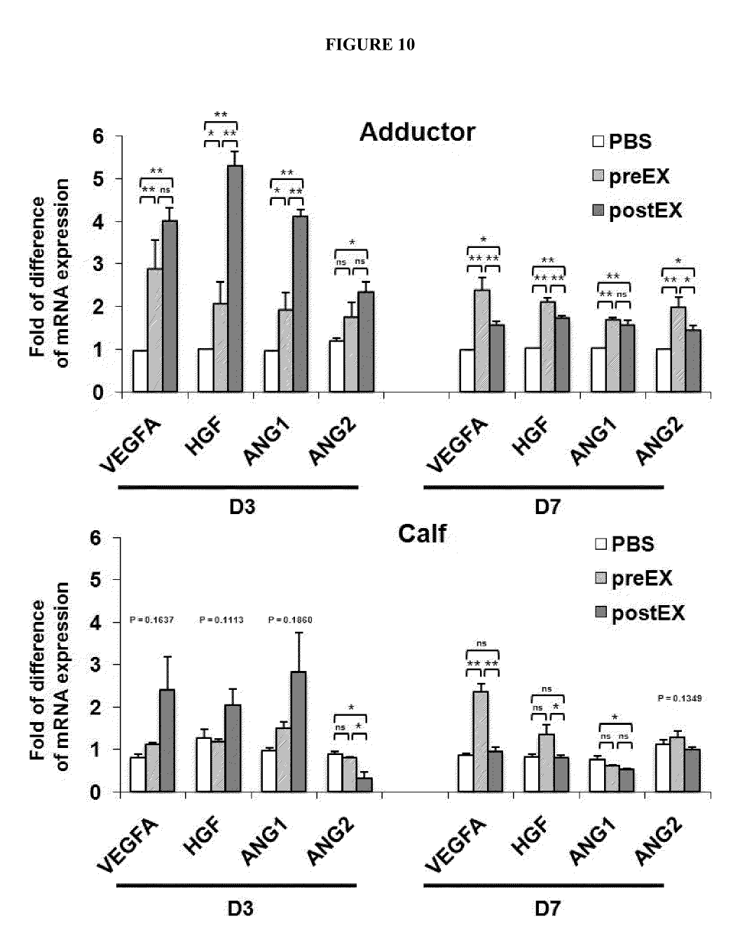

In addition, gene expressions of intrinsic angiogenic markers were significantly up regulated in the postEX-GMCD34 group especially in the adductor muscle that cells were injected locally (FIG. 10). These results were confirmed in replicate, including a set of experiments with cell treatment performed at the same time of surgery.

### Example 3

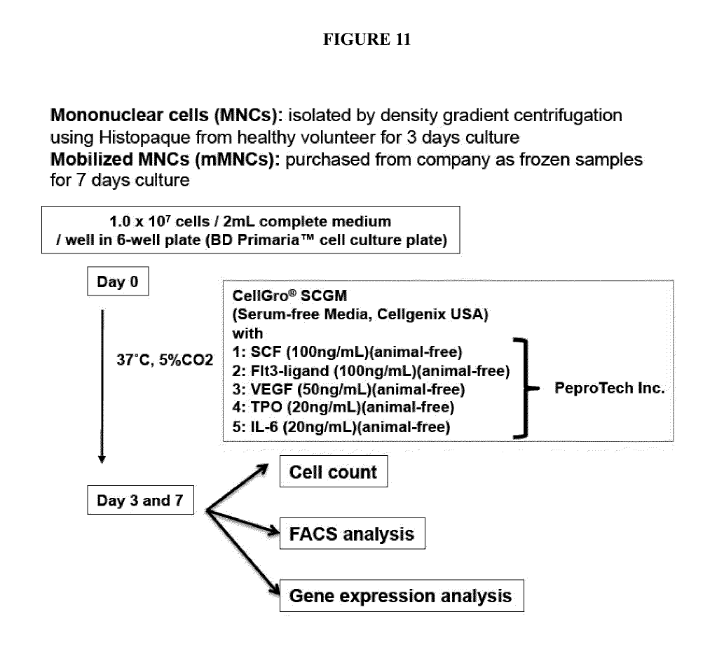

For further application of our method, we cultured human adult mononuclear cells (MNCs) and mobilized MNCs (mMNCs) with our expansion media and characterized cultured cells (FIG. 11). To isolate MNCs is extremely easier and less cost method compared with CD34⁺ cell isolation. Then, once MNCs amplification fortifying angiogenic potential with our media was confirmed, this will be a good alternative for clinical application.

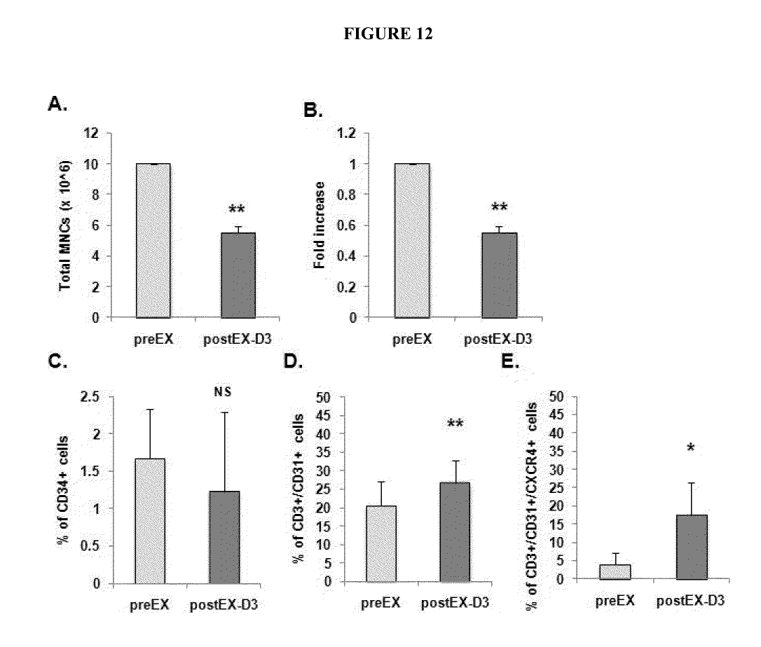

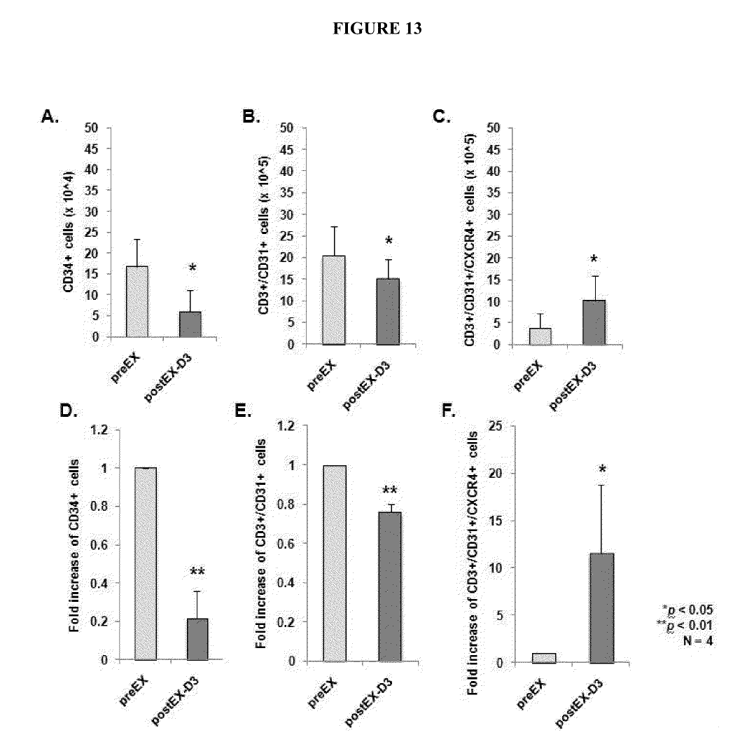

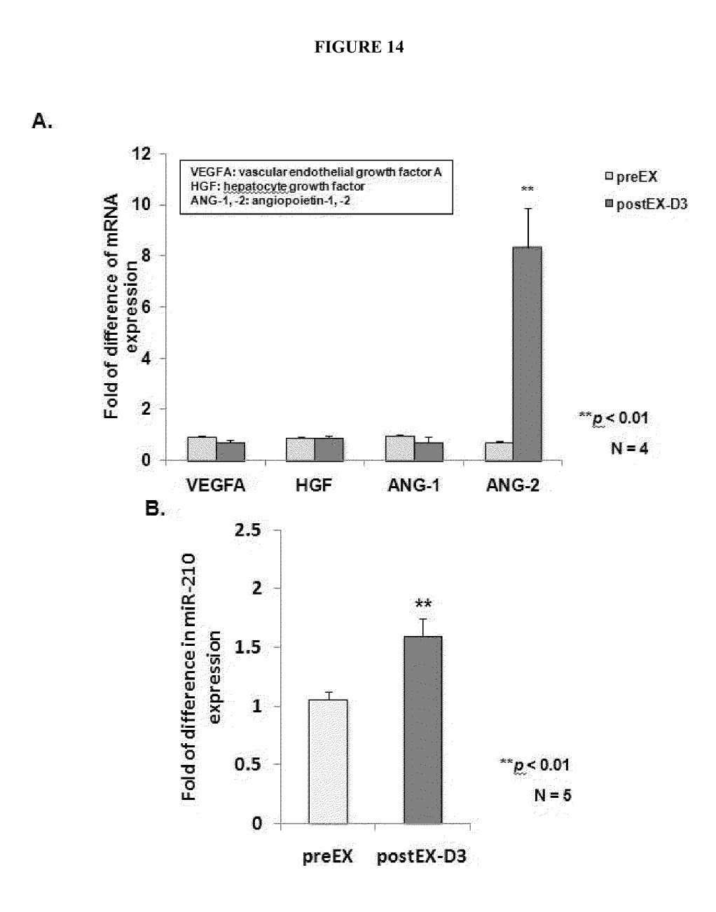

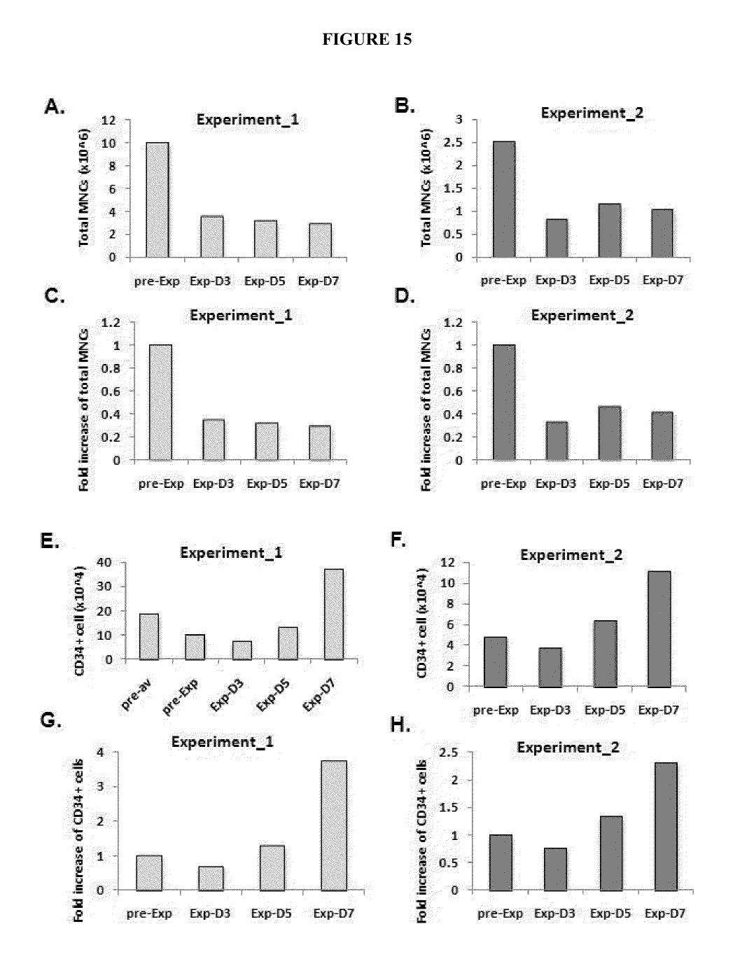

As a result of 3-day MNC culture, total MNCs and CD34⁺ cells were not expanded, however, CD3⁺/CD31⁺/CXCR4⁺ cells, known as “angiogenic T cells”, were significantly expanded (FIG. 12 and FIG. 13). Gene expression analysis showed significant up regulations of angiopoietin-2 and miR-210 (FIG. 14). After 7 days culture of mMNCs, we could expand CD34⁺ cells about 3-fold in number, although total mMNCs number was decreased (FIG. 15).

### Example 4

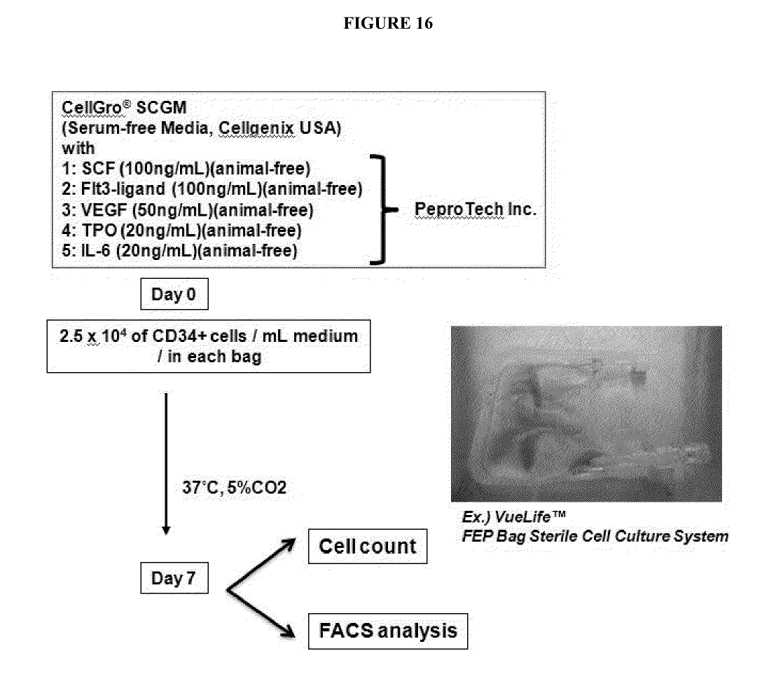

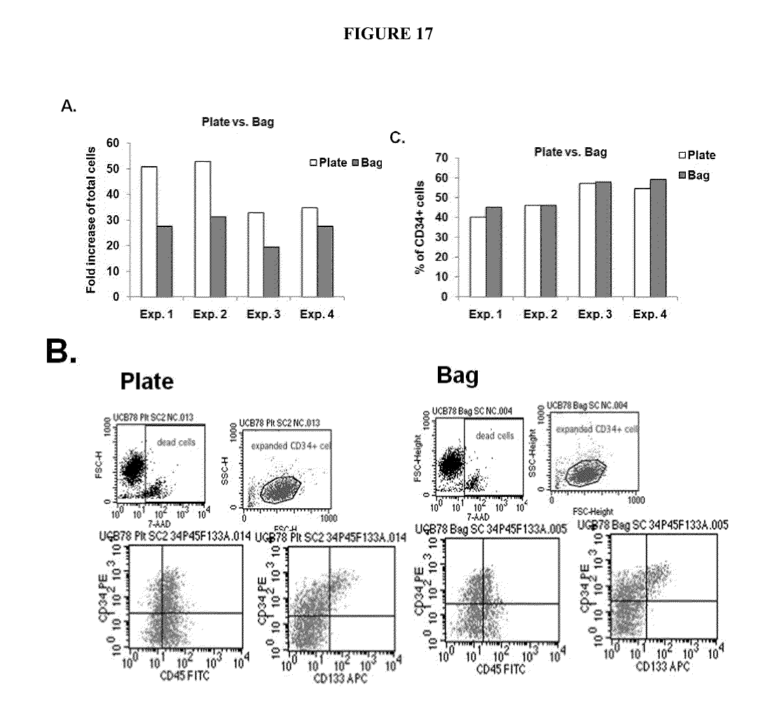

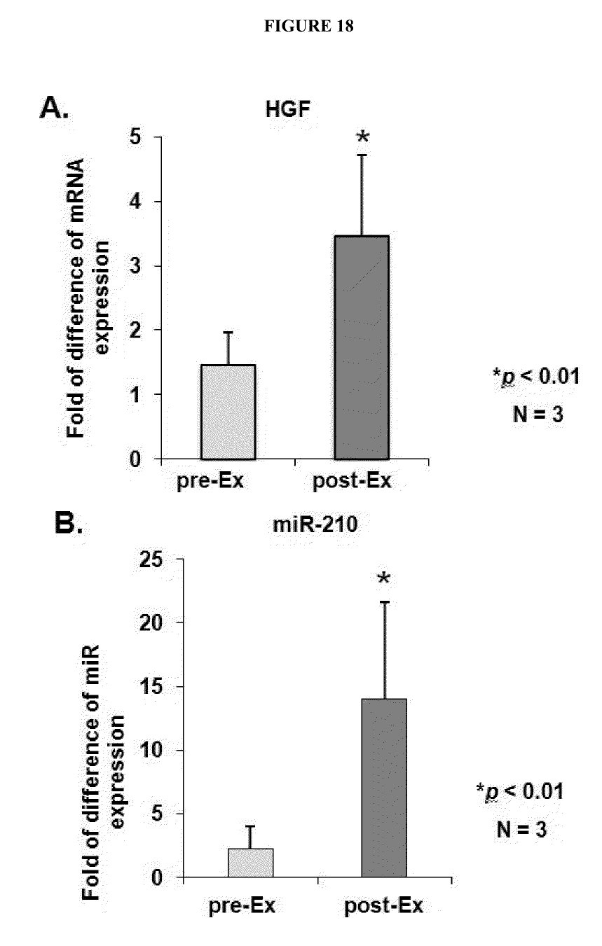

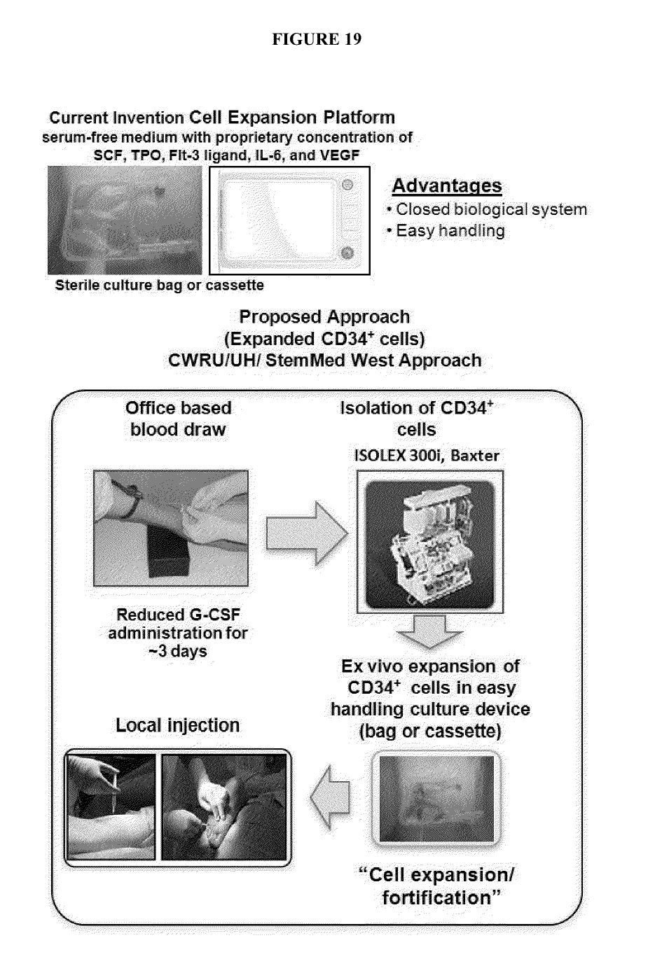

To approach more easy manipulation and less labor intension of culture for clinical application, we confirmed efficiency of our culture method using closed gas-permeable culture bag (FIG. 16). Using bag, umbilical cord blood CD34+ cells could be expanded in bag, although there was a trend lower amplification compared with conventional culture plate (FIG. 17A). The CD34-positivity showed no difference between plate and bag (FIGS. 17B and C). After 7 days expansion in bag, gene expressions of HGF and miR-210 were significantly up regulated in expanded CD34+ cells (FIGS. 18A and B). These options using closed culture devices, such as gas-permeable bag and cassette, can prevent the possible contamination and promote more feasible and stable culture procedure for cell therapy (FIG. 19).

### INDUSTRIAL APPLICABILITY

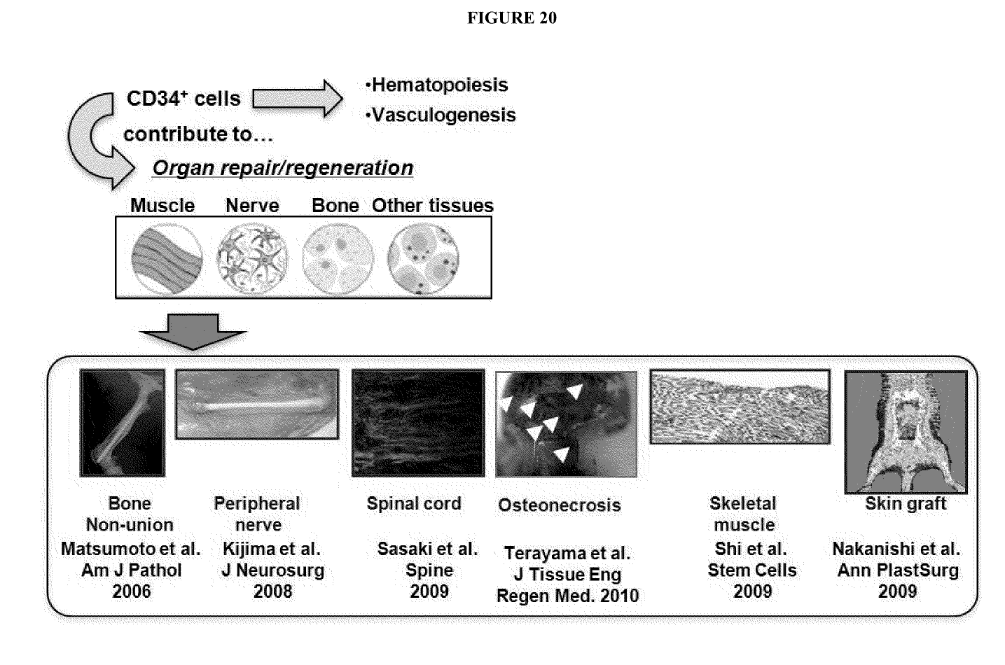

By transplantation of the cells expanded by the method of the present invention, the cardiac function (contractile function and diastolic function) in ischemic cardiac diseases was improved. In addition, expanded cells improved blood flow of ischemic limbs and reduce amputation rate. That is, the method of the present invention is considered to be useful for both qualitative and quantitative production of cells fortified angiogenic potential, and can be a useful method for a cell transplantation therapy targeting not only a vascular disorder such as ischemic disease in heart and limb but also various kind of tissue repair/regenerations via neovascularization and the like (FIG. 20).

### REFERENCES

1. Asahara, T. and Masuda, H. “Method for Amplification of Endothelial Progenitor Cell in vitro,” United States Patent Application 20080166327 (published Jul. 10, 2008).
2. Stabile, E. et al. (2003) Impaired Arteriogenic Response to Acute Hindlimb Ischemia in CD4-Knockout Mice, Circulation 108, 205-210.
3. Matsumoto, T. et al. (2006) Therapeutic potential of vasculogenesis and osteogenesis promoted by peripheral blood CD34-positive cells for functional bone healing, The American Journal of Pathology 169, 1440-1457.
4. Terayama, H. et al. (2011) Prevention of osteonecrosis by intravenous administration of human peripheral blood-derived CD34-positive cells in a rat osteonecrosis model, J Tissue Eng. Regen. Med. 5, 32-40.
5. Kijima, Y. et al. (2009) Regeneration of peripheral nerve after transplantation of CD133+ cells derived from human peripheral blood, J. Neurosurg. 110, 758-767.
6. Sasaki, H. et al. (2009) Administration of human peripheral blood-derived CD133+ cells accelerates functional recovery in a rat spinal cord injury model, Spine (Philadelphia, Pa. 1976) 34, 249-254.
7. Shi, M. et al. (2009) Acceleration of skeletal muscle regeneration in a rat skeletal muscle injury model by local injection of human peripheral blood-derived CD133-positive cells, Stem Cells 27, 949-960.
8. Nakanishi, M. et al. (2009) The effects of CD133-positive cells to a nonvascularized fasciocutaneous free graft in the rat model, Ann. Plast. Surg. 63, 331-335.
9. Asahara, T. et al. (1997) Isolation of putative progenitor endothelial cells for angiogenesis, Science 275, 964-967.
10. Asahara, T. et al. (1999) Bone Marrow Origin of Endothelial Progenitor Cells Responsible for Postnatal Vasculogenesis in Physiological and Pathological Neovascularization, Circ. Res. 85, 221-228.
11. Basak, G et al. (2009) Human embryonic stem cells hemangioblast express HLA-antigens, Journal of Translational Medicine 7, 27.
12. Loges, S. et al. (2004) Identification of the Adult Human Hemangioblast, Stem Cells Dev. 13, 229-242.

TABLE 1

| | | Approach of the Current Invention |
|---|---|---|
| | Conventional approach | (Expanded CD34⁺ cells) |
| | (fresh CD34⁺ cells) | CWRU/UH/ StemMed West |
| | ACT-34 Baxter approach | Approach |
| G-CSF | Side effects: fever, bone pain, | Reduce G-CSF dose to 3 days |
| administration/mobilization | risk of embolism, etc | Minimize Side Effects and Cost |
| | Cost: Neupogene ®, 5 μg/kg/day | |
| | x5days | |
| Aphaeresis | Invasive: central route, | No aphaeresis or Bone Marrow |
| | 12-24-hour procedure, | Aspiration |
| | specialized hospital care | Office based conventional blood |
| | Risk: large blood volume | draw (300-400 mL peripheral |
| | displacement, poor tolerability, | blood) |
| | side effects, infection, ischemia, | Significant Cost Savings |
| | embolism | |
| | Very High Costs: Hospital | |
| | charges, professional fees | |
| Quantity and Quality of cell | Limited cell number | Increased cell number |
| product | Impaired cell function | Improved cell function after 7 days |
| | (pathological background of | in StemMed West closed |
| | patients) | serum-free ex-vivo cell expansion |
| | | platform |
| | | Cost: current StemMed West |
| | | System prototype |
| | | <U$1,000/treatment; future large |
| | | commercial scale |
| | | <US$300/treatment |
| Accessibility | High complexity | Cell isolation: specialized |
| | Tertiary and specialized medical | laboratory |
| | centers | Sample collection and treatment: |
| | Highly Specialized medical | Office based approach |
| | professionals | |

We claim:

1. A method for expanding a hemangioblast population, comprising incubating hemangioblasts in serum-free culture medium, said medium comprising stem cell factor, interleukin-6, FMS-like tyrosine kinase 3, thrombopoietin, and vascular endothelial growth factor in a closed culture system under conditions such that the number of hemangioblasts increases.

2. The method of claim 1, wherein said closed culture system is selected from the group consisting of a bag, tube, flask, plate, and vessel.

3. The method of claim 1, wherein the hemangioblasts are derived from bone marrow, cord blood or peripheral blood.

4. The method of claim 1, wherein the hemangioblast is a mononuclear cell.

5. The method of claim 1, wherein the hemangioblast is CD34 positive and CD133 positive.

6. The method of claim 1, wherein the hemangioblasts are human hemangioblasts.

7. The method of claim 1, wherein the serum-free culture medium further comprises a transforming growth factor β inhibitor.

8. An endothelial progenitor cell obtained by the method of claim 1.

9. The method of claim 1, wherein said hemangioblast is obtained from a subject treated with:
a) granulocyte colony stimulating factor over 3 days or less.

10. The method of claim 9, wherein, following said treating, a peripheral blood sample is obtained from said subject.

11. The method of claim 10, wherein said blood sample is subjected to density gradient centrifugation in order to obtain said hemangioblasts.

12. The method of claim 10, wherein said blood sample is 400 milliliters or less in volume.

13. The method of claim 9, wherein said subject is a human.

14. A composition comprising an endothelial progenitor cell obtained by the method of claim 1, wherein said cell is substantially free of a biogenic substance derived from an animal of a different species from the animal, from which the endothelial progenitor cell is derived.

15. A kit for preparing a serum-free culture medium, said kit comprising stem cell factor, interleukin-6, FMS-like tyrosine kinase 3, thrombopoietin, vascular endothelial growth factor, and serum-free culture medium in a closed culture system.

16. A method for culturing a hemangioblast, comprising incubating the hemangioblast in a closed culture system in serum-free culture medium containing stem cell factor, interleukin-6, FMS-like tyrosine kinase 3, thrombopoietin, and vascular endothelial growth factor.

17. A method of treating a blood donor in order to obtain hemangioblasts from said donor provided:
a) treating said donor with granulocyte colony stimulating factor over the course of 3 or less days,
b) extracting a peripheral blood sample from said donor after the course of treatment, and
c) isolating desired mononuclear cells by density gradient centrifugation.

18. The method of claim 17, wherein said blood sample is 400 milliliters or less in volume.

19. The method of claim 17, wherein said subject is a human.
